(19) United States
(12) Patent Application Publication
(10) Pub. No.: US 2011/0315659 A1
(43) Pub. Date: Dec. 29, 2011

(54) PATTERN FORMATION METHOD AND IMPRINT MATERIAL

(76) Inventors: Yusuke SEKIGUCHI, Kanagawa-ken (JP); Yoshihisa Kawamura, Kanagawa-ken (JP)

(21) Appl. No.: 13/168,417

(22) Filed: Jun. 24, 2011

(30) Foreign Application Priority Data
Jun. 28, 2010 (JP) 2010-146187

(51) Int. Cl. B05D 3/10; C07D 233/60; C23F 1/00

(52) U.S. Cl. 216/37; 548/334.1

(57) ABSTRACT

According to one embodiment, a pattern formation method is disclosed. The method can include forming a foundation film on a patterning film. The foundation film includes a reaction initiator to produce at least one of an acid and a base. The method can include forming an imprint film having an uneven configuration by coating an imprint material onto the foundation film and by causing a template to contact the imprint material. The method can include increasing an etching rate of a first portion of the imprint film higher than a second portion by introducing the at least one of acid and base into the first portion. The first portion is on the foundation film side. The second portion is a portion excluding the first portion. The method can include patterning the patterning film using a protruding portion of the uneven configuration as a mask.

### CROSS-REFERENCE TO RELATED APPLICATIONS

This application is based upon and claims the benefit of priority from the prior Japanese Patent Application No. 2010-146187, filed on Jun. 28, 2010; the entire contents of which are incorporated herein by reference.

### FIELD

Embodiments described herein relate generally to a pattern formation method and an imprint material.

### BACKGROUND

Nanoimprinting used to transfer a master form onto a substrate is drawing attention as a technology to form ultra-fine patterns with high productivity when manufacturing electronic devices having ultra-fine structures such as semiconductor devices, MEMS (Micro Electro Mechanical System) devices, etc.

In nanoimprinting, a pattern is transferred onto a resin on the substrate by bringing the master form (the template) having the pattern to be transferred into contact with the resin on the substrate and by curing the resin.

When processing is performed in nanoimprinting to remove the residual film of the resin material that occurs between the protruding portion of the template and the substrate, the height of the protruding portion of the transfer pattern is reduced and the patternability of the substrate using the transfer pattern as a mask degrades.

### BRIEF DESCRIPTION OF THE DRAWINGS

FIG. 1 is a flowchart illustrating a pattern formation method according to a first embodiment;

FIG. 2A to FIG. 2H are schematic cross-sectional views illustrating the pattern formation method according to the first embodiment;

FIG. 3A to FIG. 3G are schematic cross-sectional views illustrating a pattern formation method of a reference example;

FIG. 4A to FIG. 4H are schematic cross-sectional views illustrating another pattern formation method according to the first embodiment;

FIG. 5 is a flowchart illustrating a pattern formation method according to a second embodiment;

FIG. 6A to FIG. 6H are schematic cross-sectional views illustrating the pattern formation method according to the second embodiment; and

FIG. 7A to FIG. 7H are schematic cross-sectional views illustrating another pattern formation method according to the second embodiment.

### DETAILED DESCRIPTION

In general, according to one embodiment, a pattern formation method is disclosed. The method can include forming a foundation film on a patterning film. The foundation film includes a reaction initiator configured to produce at least one selected from an acid and a base. The method can include forming an imprint film having an uneven configuration by coating an imprint material onto the foundation film and by causing a template to contact the imprint material. The method can includes increasing an etching rate of a first portion of the imprint film to be higher than an etching rate of a second portion of the imprint film by causing the at least one selected from the acid and the base to be produced from the reaction initiator and by introducing the at least one selected from the acid and the base into the first portion. The first portion is a portion of the imprint film on the foundation film side. The second portion is a portion of the imprint film excluding the first portion. The method can includes removing a recessed portion of the uneven configuration of the imprint film. The method can includes patterning the patterning film using a protruding portion of the uneven configuration of the imprint film as a mask.

Embodiments of the invention will now be described with reference to the drawings.

The drawings are schematic or conceptual; and the relationships between the thicknesses and widths of portions, the proportions of sizes among portions, etc., are not necessarily the same as the actual values thereof. Further, the dimensions and the proportions may be illustrated differently among the drawings, even for identical portions.

In the specification and the drawings of the application, components similar to those described in regard to a drawing thereinabove are marked with like reference numerals, and a detailed description is omitted as appropriate.

### First Embodiment

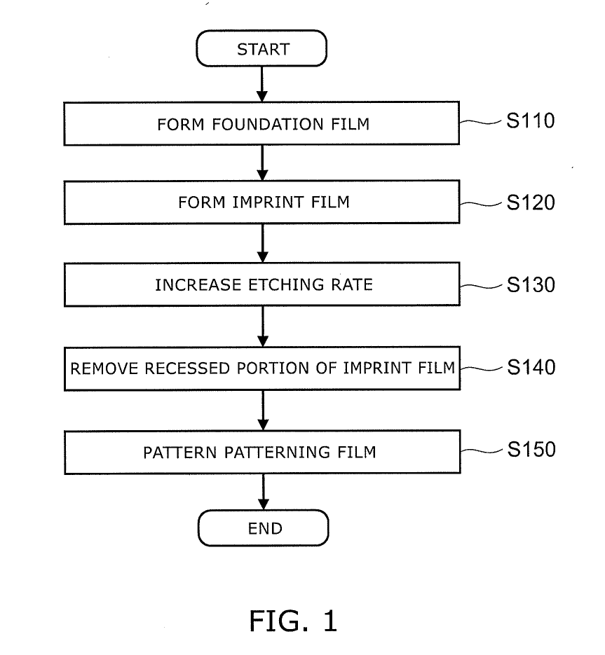

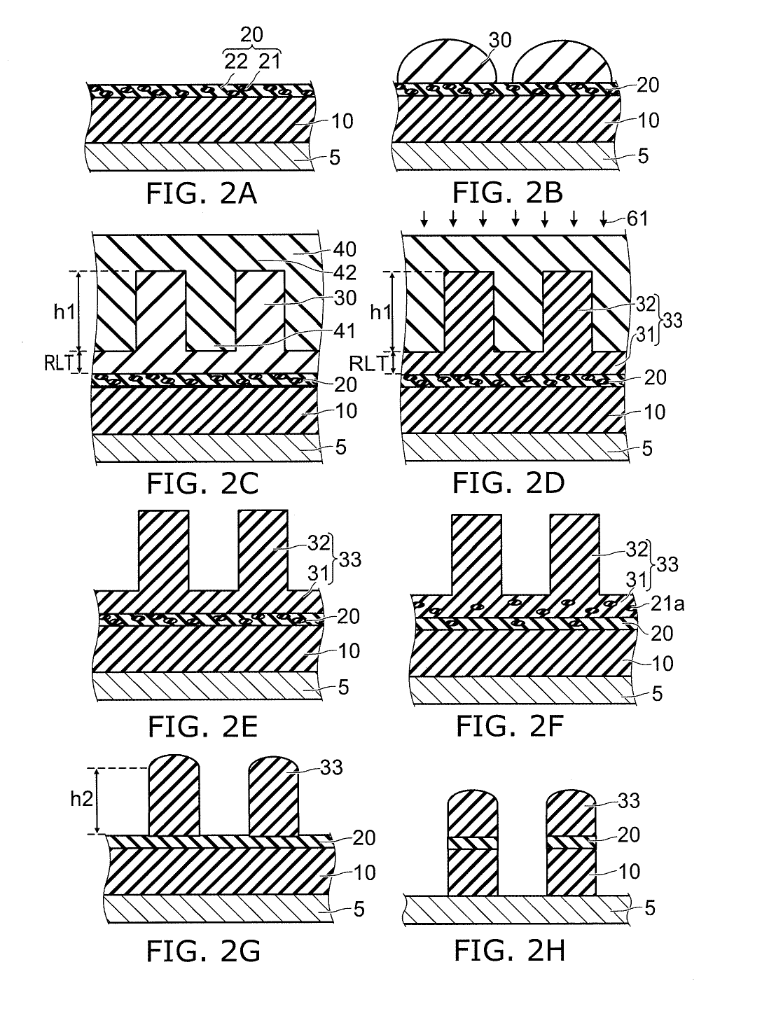

FIG. 1 is a flowchart illustrating a pattern formation method according to a first embodiment. FIG. 2A to FIG. 2H are schematic cross-sectional views illustrating the pattern formation method according to the first embodiment.

In the pattern formation method according to this embodiment as illustrated in FIG. 1 and FIG. 2A, a foundation film 20 is formed on a patterning film 10, where the foundation film 20 includes a reaction initiator 21 configured to produce at least one selected from an acid and a base (step S110).

The patterning film 10 is provided on a substrate 5 including, for example, a semiconductor such as silicon, various insulators, various conductors (including metals), etc. Or, the patterning film 10 may be the substrate 5 itself. Hereinbelow, the case is described where the patterning film 10 is provided on the substrate 5.

A semiconductor film such as silicon, various insulating films, various conductor films (including metal films), for example, may be used as the patterning film 10. Hereinbelow, the case is described where a silicon oxide film with a thickness of 40 nanometers (nm) is used as the patterning film 10.

The reaction initiator 21 may include, for example, a material configured to produce an acid by heating, a material configured to produce an acid by irradiating light, a material configured to produce a base by heating, and a material configured to produce a base by irradiating light, etc.

Hereinbelow, the case is described where an aromatic sulfonium salt configured to produce an acid by heating is used as the reaction initiator 21.

The foundation film 20 may include one other material 22 in addition to the reaction initiator 21 recited above. The one other material 22 may include a solvent for stabilizing the material used to form the foundation film 20 including the reaction initiator 21 as a solution, etc. Also, the one other material 22 includes a material for increasing the adhesion of the patterning film 10. In other words, the foundation film 20 may include an adhesion improver other than the reaction initiator 21.

The foundation film 20 is, for example, spin-coated onto the patterning film 10. The thickness of the foundation film 20 is, for example, not less than 1 nm and not more than 5 nm, e.g., 3 nm. However, the embodiment is not limited thereto. The formation method of the foundation film 20 and the thickness of the foundation film 20 are arbitrary.

As illustrated in FIG. 1, the imprint material is coated onto the foundation film 20; and the imprint film having the uneven configuration is formed by causing a template to contact the imprint material (step S120). An imprint film 33 is made of the imprint material and has a bottom portion and an apical portion that reflect the uneven configuration of the template.

In other words, as illustrated in FIG. 2B, an imprint material 30 is coated onto the foundation film 20. The imprint material 30 includes a material having an etching rate that increases by at least one selected from an acid and a base produced from the reaction initiator 21 included in the foundation film 20. The imprint material 30 includes, for example, a photocurable acrylic resin, etc.

An inkjet method, etc., for example, may be used to coat the imprint material 30. The amount of one drop of the imprint material 30 coated using the inkjet method is, for example, 10 picoliters (pi). However, the embodiment is not limited thereto. The coating method and the coating amount of the imprint material 30 are arbitrary.

As illustrated in FIG. 2C, the imprint material 30 is caused to contact a template 40. For example, the template 40 may include a material that transmits light in the case where the imprint material 30 is photosensitive (e.g., photocurable). The template 40 may include, for example, quartz and the like. However, the embodiment is not limited thereto. The material of the template 40 is arbitrary.

An unevenness is provided in the transfer surface (the surface on the side contacting the imprint material 30) of the template 40. In other words, the template 40 includes a template protrusion 41 and a template recess 42. Here, a height h1 of the template protrusion 41 (a depth of the template recess 42) is, for example, 65 nm. The width of the template protrusion 41 is, for example, 40 nm; and the width of the template recess 42 is, for example, 40 nm. However, the height h1 of the template protrusion 41, the width of the template protrusion 41, and the width of the template recess 42 are arbitrary.

By causing the template 40 to contact the imprint material 30, the imprint material 30 enters the template recess 42 of the template 40; and the imprint material 30 deforms into a configuration conforming to the configuration of the template protrusion 41 and the template recess 42. At this time, the distance between the template protrusion 41 and the patterning film 10 is not zero; and a prescribed spacing is provided between the template protrusion 41 and the patterning film 10. The imprint material 30 is provided between the template protrusion 41 and the patterning film 10; and this becomes a residual film. A residual film thickness RLT (Residual Layer Thickness), which is the thickness of the residual film, is controlled by, for example, the amount (the amount per unit surface area) of the imprint material 30 coated onto the patterning film 10. The residual film thickness RLT is, for example, 30 nm.

As illustrated in FIG. 2D, the imprint material 30 is cured by irradiating, for example, light 61 through the template 40 onto the imprint material 30. Thereby, the imprint film 33 made of the imprint material 30 and including a bottom portion 31 and an apical portion 32 that reflect the uneven configuration of the template 40 is formed. The imprint film 33 includes the bottom portion 31 and the apical portion 32 that correspond to the template protrusion 41 and the template recess 42. The thickness of the bottom portion 31 of the imprint film 33 substantially corresponds to the residual film thickness RLT.

Thus, the formation process of the imprint film 33 (step S120) may include the process of curing the imprint material 30 by irradiating the light 61 onto the imprint material 30 in the state in which the template 40 is caused to contact the imprint material 30. However, the embodiment is not limited thereto. The method of curing the imprint material 30 is arbitrary. For example, the formation process of the imprint film 33 (step S120) may include a process of curing the imprint material 30 by heating the imprint material 30 in the state in which the template 40 is caused to contact the imprint material 30.

Subsequently, as illustrated in FIG. 2E, the template 40 is released by being released from the cured imprint film 33.

After the releasing of the template 40 as illustrated in FIG. 1, at least one selected from an acid and a base is caused to be produced from the reaction initiator 21; the at least one selected from the acid and the base is introduced into a portion (a first portion) of the imprint film 33 on the foundation film 20 side; and the etching rate of the portion of the imprint film 33 having the at least one selected from the acid and the base introduced is increased to be higher than the etching rate of a portion (a second portion) excluding the portion of the imprint film 33 recited above (step S130).

In other words, the at least one selected from the acid and the base produced is introduced into the bottom portion 31 of the imprint film 33; and a reaction is implemented to increase the etching rate of the bottom portion 31 to be higher than the etching rate of the apical portion 32.

Specifically, as illustrated in FIG. 2F, an acid 21a is caused to be produced from the reaction initiator 21 inside the foundation film 20 by heating the foundation film 20; and the acid 21a is introduced into the bottom portion 31 of the imprint film 33 by diffusing the acid 21a into the bottom portion 31 of the imprint film 33. For example, the acid 21a is produced from the reaction initiator 21 by heating the patterning body for 1 minute at a temperature of 150° C. in air. The acid 21a is diffused into the bottom portion 31 by this heating.

In the bottom portion 31 of the imprint film 33, the imprint film 33 reacts with water inside the air with the introduced acid 21a as a catalyst. For example, the resin included in the imprint film 33 has ester bonds; the ester bonds undergo hydrolysis due to the acid 21a; and carboxylic acid and alcohol, for example, are produced. Thereby, the ester bonds inside the resin are broken. On the other hand, the acid 21a is not introduced into the apical portion 32. Thereby, the etching rate of the bottom portion 31 is higher than the etching rate of the apical portion 32.

Thus, the reaction process recited above (step S130) includes a heating process of heating the foundation film 20 to cause the at least one selected from the acid and the base to be produced and cause the at least one selected from the acid and the base produced to move into the imprint film 33. Specifically, the at least one selected from the acid and the base produced is moved into the bottom portion 31 of the imprint film 33.

After the reaction process as illustrated in FIG. 1, the recessed portion of the imprint film 33 is removed (step S140). In other words, the bottom portion 31 is removed by etching the imprint film 33.

In other words, as illustrated in FIG. 2G, anisotropic dry etching such as, for example, oxygen plasma is performed to remove the bottom portion 31 of the imprint film 33. At this time, the decrease of the height of the apical portion 32 of the imprint film 33 is small because the etching rate of the apical portion 32 is lower than that of the bottom portion 31.

For example, the etching rate of the bottom portion 31 is 120 nm/min (nanometer/minute); and the etching rate of the apical portion 32 is about 90 nm/min.

The cross-sectional area of the corner of the imprint film 33 at the tip of the apical portion 32 may be reduced by being etched during the patterning; and the height of the portion of the apical portion 32 having a constant cross-sectional area is taken as an apical portion height h2. The apical portion height h2 corresponds to the effective thickness of the imprint film 33 that functions as the mask. Therefore, the film thickness (the apical portion height h2) of the imprint film 33 which can be used as the mask of the actual patterning is smaller than the height h1 of the template protrusion 41.

For example, the apical portion height h2 is 38 nm in the case where the height h1 of the template protrusion 41 (the depth of the template recess 42) is 65 nm and the thickness RLT of the bottom portion 31 is 30 nm.

As illustrated in FIG. 1 and FIG. 2H, the patterning film 10 is patterned using the protruding portion of the imprint film 33 as a mask (step S150). In other words, the foundation film 20 is patterned and the patterning film 10 (in this example, the silicon oxide film) is patterned by RIE with, for example, a CF-based gas using the remaining portion of the imprint film 33 as a mask. Although the thickness of the imprint film 33 used as the mask also is reduced during the patterning of the patterning film 10 to the desired thickness, in this embodiment, the imprint film 33 can exist until the end of the patterning of the patterning film 10 and the desired patterning of the patterning film 10 is possible because the apical portion height h2 is high.

For example, the patterning selectivity of the imprint film 33 and the patterning film 10 in the patterning of the patterning film 10 (in this case, the silicon oxide film) is 1.3. Therefore, it is possible to pattern a silicon oxide film having a thickness not more than about 50 nm.

After the patterning of the patterning film 10, oxygen plasma processing is performed; and the imprint film 33 is removed by ashing. Thereby, the pattern of the silicon oxide film is formed.

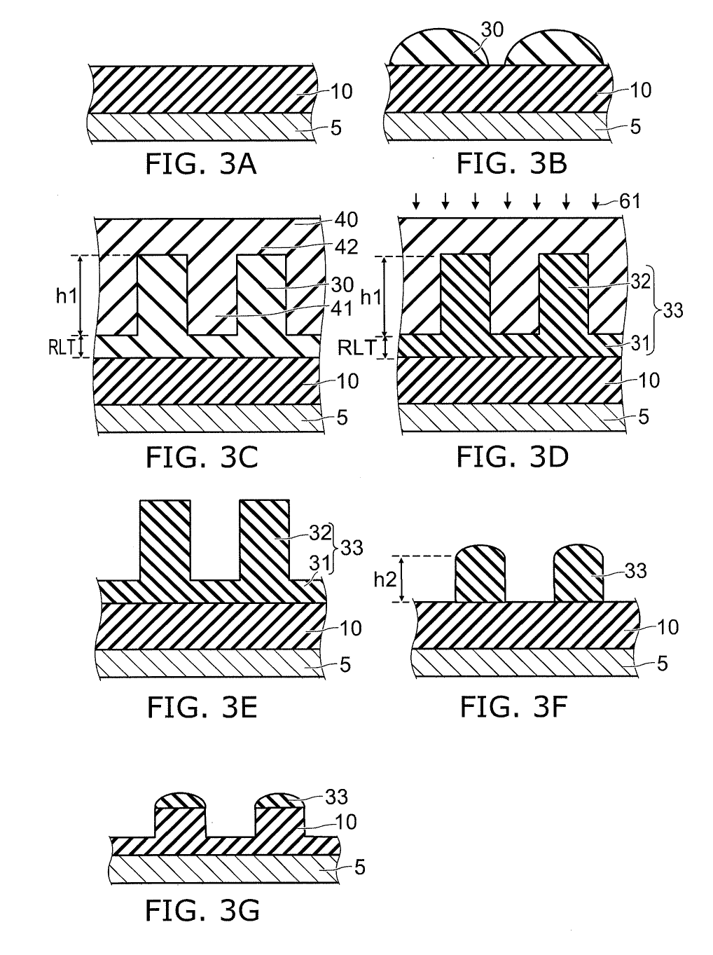

FIG. 3A to FIG. 3G are schematic cross-sectional views illustrating a pattern formation method of a reference example.

The foundation film 20 is not formed in the pattern formation method of the reference example.

In other words, as illustrated in FIGS. 3A and 3B, the imprint material 30 is coated onto the patterning film 10. Then, as illustrated in FIG. 3C, the template 40 is caused to contact the imprint material 30. Then, as illustrated in FIG. 3D, the imprint film 33, which is made of the imprint material 30 and includes the bottom portion 31 and the apical portion 32 that reflect the uneven configuration of the template 40, is formed.

Continuing as illustrated in FIG. 3E, the template 40 is released; and the bottom portion 31 is removed by etching the imprint film 33 as illustrated in FIG. 3F. At this time, in the reference example, the etching rate of the bottom portion 31 is the same as the etching rate of the apical portion 32. For example, both the etching rate of the bottom portion 31 and the etching rate of the apical portion 32 are about 90 nm/min.

Therefore, the height of the apical portion 32 is reduced by an amount corresponding to the thickness of the bottom portion 31 when removing the bottom portion 31. The apical portion height h2 of the reference example is, for example, 25 nm in the case where the height h1 of the template protrusion 41 (the depth of the template recess 42) is 65 nm and the thickness RLT of the bottom portion 31 is 30 nm, where the apical portion height h2 is the height of the portion of the apical portion 32 having a constant cross-sectional area.

As illustrated in FIG. 3G, although the patterning film 10 is patterned using the imprint film 33 as a mask, the imprint film 33 undesirably vanishes partway through the patterning of the patterning film 10 and the patterning of the patterning film 10 cannot be performed because the height (the apical portion height h2) of the imprint film 33 is low.

Thus, in the pattern formation method of the reference example, there are many constraints on the thickness and the material of the patterning film 10; and it is difficult to pattern the patterning film 10 of the desired thickness and the desired material. Moreover, in the pattern formation method of the reference example, in the case where the height h1 of the template protrusion 41 (the depth of the template recess 42) is increased and the aspect ratio of the unevenness is increased, the formation of the template 40 becomes difficult, the releasability of the transfer process worsens, and it is easy for destruction of the transferred imprint film 33 to occur.

Conversely, by using the foundation film 20 in the pattern formation method according to this embodiment, the bottom portion 31 of the imprint film 33 can be selectively removed efficiently; and a high apical portion height h2 of the apical portion 32 of the imprint film 33 can be maintained. Thereby, the constraints on the thickness and the material of the patterning film 10 are relaxed; and it is possible to pattern a patterning film 10 of the desired thickness and the desired material. According to the pattern formation method according to this embodiment, a pattern formation method capable of efficiently removing the residual film can be provided.

As another reference example, it is conceivable to use a method in which an imprint material including a depolymerizing polymer containing an acid-producing material is coated onto the substrate surface; the protruding portion of the heated template is closely adhered to the imprint material; the acid is produced by the heat of the protruding portion; and the depolymerizing polymer closely adhered to the protruding portion is depolymerized. However, it is necessary to control the height with exceedingly high precision to bring only the protruding portion of the heated template near the imprint material; and practical problems exist. Also, it is difficult to locally heat only the protruding portion of the template; the temperature of the entire template including the recessed portion undesirably increases when heating the protruding portion; and it is difficult to practically depolymerize only the depolymerizing polymer of the portion closely adhered to the protruding portion. Moreover, partially heating the template causes distortion of the template to occur and reduces the precision.

As yet another reference example, it is conceivable to use a method that uses a template in which the surface of the protruding portion of the template has an acidic portion. However, in such a case as well, it is difficult to form the acidic portion selectively in the surface of the protruding portion of the template; and practical problems exist concerning the life of the template as well.

Conversely, in the pattern formation method according to this embodiment, it is unnecessary to control the height with high precision; local heating and local surface processing of the template are unnecessary; and the efficient removal of the residual film is practically possible.

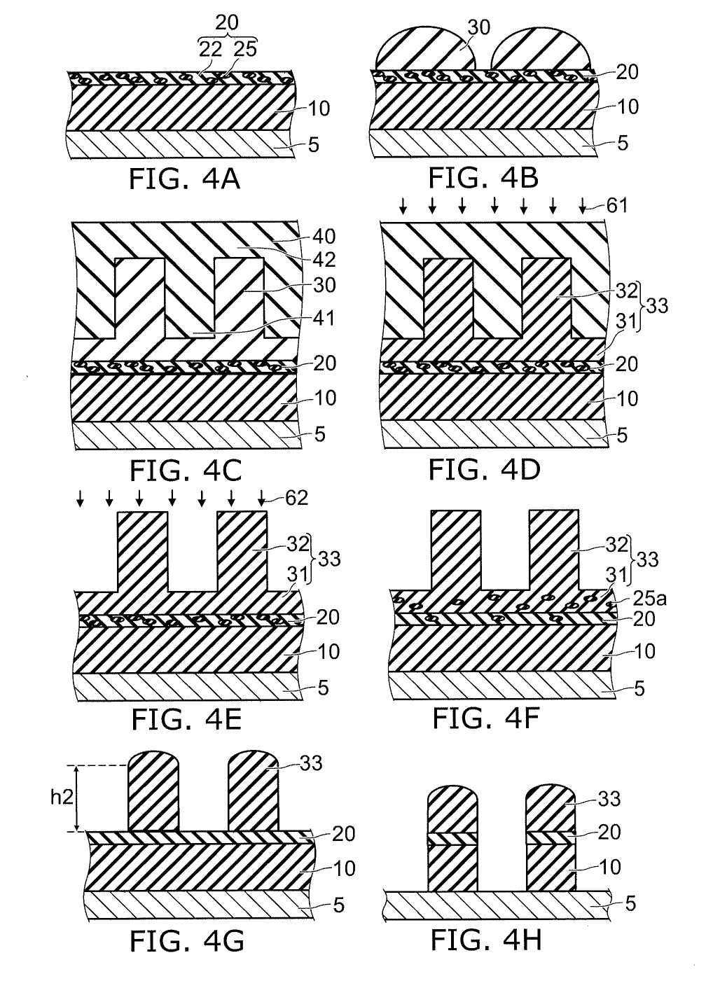

FIG. 4A to FIG. 4H are schematic cross-sectional views illustrating another pattern formation method according to the first embodiment.

In this pattern formation method according to this embodiment, a substance that produces the at least one selected from the acid and the base by being irradiated with light is used as the reaction initiator.

As illustrated in FIG. 4A, the foundation film 20 including a reaction initiator 25 that produces the at least one selected from the acid and the base by being irradiated with light is formed on the patterning film 10 (step S110).

Bis(t-butylsulfonyl)diazomethane, for example, is used as the reaction initiator 25. In such a case as well, the foundation film 20 may include one other material 22 including a material for improving the adhesion, etc., in addition to the reaction initiator 25 recited above. The thickness of the foundation film 20 is, for example, 3 nm. In such a case as well, the formation method of the foundation film 20 and the thickness of the foundation film 20 are arbitrary.

As illustrated in FIG. 4B, the imprint material 30 is coated onto the foundation film 20. The imprint material 30 may include, for example, a photocurable acrylic resin, etc.

As illustrated in FIG. 4C, the template 40 is caused to contact the imprint material 30. The height h1 of the template protrusion 41 (the depth of the template recess 42) is, for example, 65 nm. The width of the template protrusion 41 is, for example, 40 nm; and the width of the template recess 42 is, for example, 40 nm. In such a case as well, the residual film thickness RLT which is the thickness of the residual film is, for example, 30 nm.

As illustrated in FIG. 4D, the imprint material 30 is cured by irradiating the light 61 through the template 40 onto the imprint material 30. The wavelength of the light 61 is, for example, 365 nm. Thereby, the imprint film 33, which is made of the imprint material 30 and includes the bottom portion 31 and the apical portion 32 that reflect the uneven configuration of the template 40, is formed. In other words, step S120 is implemented.

As illustrated in FIG. 4E, the template 40 is released by being released from the cured imprint film 33.

As illustrated in FIG. 4F, light 62 is irradiated onto the foundation film 20; an acid 25a is produced from the reaction initiator 25 inside the foundation film 20; the acid 25a is diffused into the bottom portion 31 of the imprint film 33; and the acid 25a is introduced into the bottom portion 31 of the imprint film 33. The light 62 at this time has a wavelength of, for example, not more than 270 nm. Specifically, the wavelength of the light 62 may be 248 nm.

Subsequently, the acid 25a produced from the reaction initiator 25 is diffused into the bottom portion 31 by heating the patterning body for 1 minute at a temperature of 110° C. in air.

The bottom portion 31 of the imprint film 33 is decomposed by the acid 25a. On the other hand, the acid 25a is not introduced into the apical portion 32. In other words, a reaction (e.g., a hydrolysis reaction) is implemented in the bottom portion 31 such that the etching rate of the bottom portion 31 becomes higher than the etching rate of the apical portion 32. In other words, step S130 is implemented.

As illustrated in FIG. 4G, anisotropic dry etching using, for example, oxygen plasma, etc., is performed to remove the bottom portion 31 of the imprint film 33. In other words, step S140 is implemented.

The decrease of the height of the apical portion 32 of the imprint film 33 is small because the etching rate of the apical portion 32 is lower than that of the bottom portion 31. For example, the etching rate of the bottom portion 31 is 120 nm/min; and the etching rate of the apical portion 32 is about 90 nm/min.

For example, the apical portion height h2 is 38 nm in the case where the height h1 of the template protrusion 41 (the depth of the template recess 42) is 65 nm and the thickness RLT of the bottom portion 31 is 30 nm.

As illustrated in FIG. 4H, the patterning film 10 is patterned using the imprint film 33 as a mask. In other words, step S150 is implemented. Although the thickness of the imprint film 33 used as the mask also is reduced during the patterning of the patterning film 10 of the desired thickness, in this embodiment, the imprint film 33 can exist until the end of the patterning of the patterning film 10 and the desired patterning of the patterning film 10 is possible because the apical portion height h2 is high.

After the patterning of the patterning film 10, oxygen plasma processing is performed; and the imprint film 33 is removed by ashing. Thereby, the pattern of the silicon oxide film is formed.

Thus, in this specific example, the reaction process (step S130) recited above includes a light irradiation process of irradiating the light 62 onto the foundation film 20 to produce the at least one selected from the acid and the base and a heating process of heating the foundation film 20 to cause the at least one selected from the acid and the base to move into the imprint film 33. Specifically, the at least one selected from the acid and the base is moved into the bottom portion 31.

The wavelength of the light 62 of the light irradiation process recited above can be shorter than the wavelength of the light 61 irradiated onto the imprint material 30 in the formation of the imprint film 33 (step S120). Thereby, the undesirable occurrence in the process of forming the imprint film 33 of the at least one selected from the acid and the base being produced from the reaction initiator 25 and entering into the uncured imprint material 30 to reduce the etching rate of the apical portion 32 of the imprint material 30 which needs to be cured is suppressed; and the desired patterning is easier.

### Second Embodiment

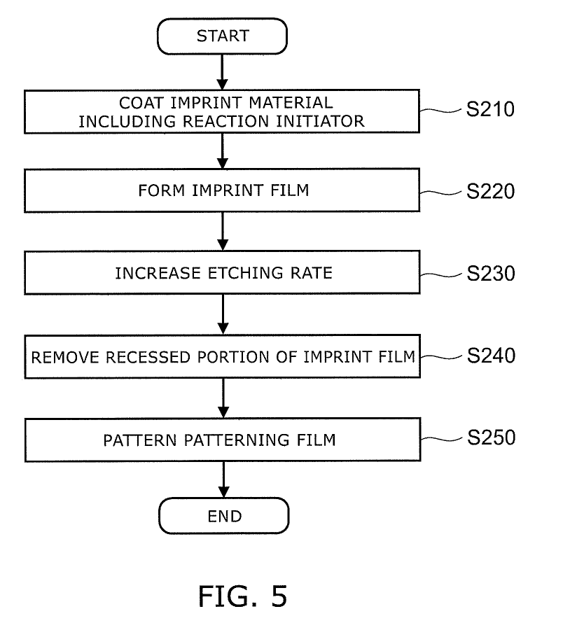

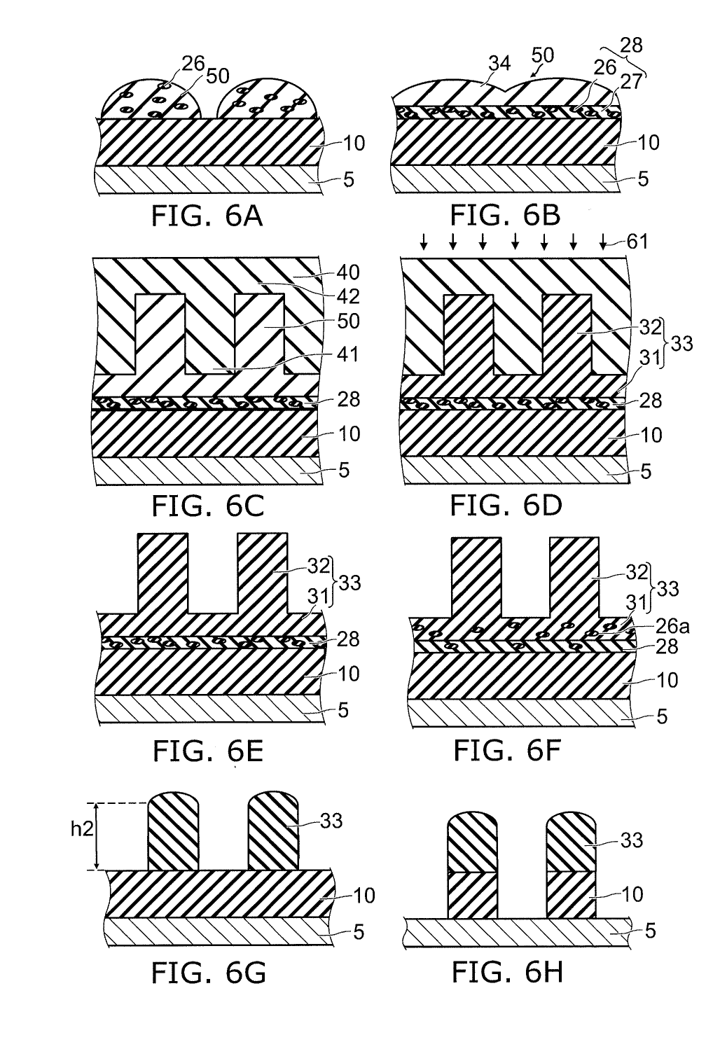

FIG. 5 is a flowchart illustrating a pattern formation method according to a second embodiment. FIG. 6A to FIG. 6H are schematic cross-sectional views illustrating the pattern formation method according to the second embodiment.

In the pattern formation method according to this embodiment as illustrated in FIG. 5 and FIG. 6A, an imprint material 50 including a reaction initiator 26 configured to produce at least one selected from an acid and a base is coated onto the patterning film 10 (step S210). An aromatic sulfonium salt, for example, which is configured to produce an acid by heating, may be used as the reaction initiator 26.

Then, as illustrated in FIG. 5, the imprint film 33 having an uneven configuration is formed by causing the template 40 to contact the imprint material 50 in a state in which the reaction initiator 26 is localized in a lower portion of the imprint material 50 on the patterning film 10 side (step S220). The imprint film 33 includes the imprint material 50 and includes the bottom portion 31 and the apical portion 32 that reflect the uneven configuration of the template 40.

In other words, as illustrated in FIG. 6B, the imprint material 50 coated onto the patterning film 10 is separated into a lower portion 28 and an upper portion 34. The lower portion 28 is the portion of the imprint material 50 on the patterning film 10 side. The concentration of the reaction initiator 26 of the lower portion 28 is higher than the concentration of the reaction initiator 26 of the upper portion 34. In other words, the reaction initiator 26 is localized in the lower portion 28. The lower portion 28 and the upper portion 34 such as those recited above are separated by, for example, adjusting the surface tension of the components included in the imprint material 30. Other than the reaction initiator 26, the lower portion 28 may include one other material 27 such as a material for increasing the adhesion strength.

For example, a substance having a surfactant structure is used as the reaction initiator 26. A surfactant structure is a structure having a hydrophilic portion chemically bonded to a hydrophobic portion. In the case where such a reaction initiator 26 is used and the surface of the patterning film 10 is, for example, hydrophilic (having a high surface tension), the concentration of the reaction initiator 26 in the portion of the coated imprint material 50 on the lower side inside the film (the portion on the surface side of the patterning film 10) is high; and the concentration of the reaction initiator 26 in the portion on the upper side inside the film is low. Thereby, the reaction initiator 26 can be localized in the lower portion 28. Thus, layer separation is performed. In this specific example, the patterning film 10 is a silicon oxide film; and the surface of the patterning film 10 is made hydrophilic by silanolizing. Even in the case where the patterning film 10 is not a silicon oxide film, the surface of the patterning film 10 can be hydrophilized (made to have a high surface tension) by performing processing suited to the patterning film 10 used.

As illustrated in FIG. 6C, the template 40 is caused to contact the imprint material 50 in a state in which the reaction initiator 26 is localized in the lower portion 28 of the imprint material 50 on the patterning film 10 side.

A low surface-tension processing such as, for example, fluorination is performed on the surface of the template 40. Therefore, the movement of the reaction initiator 26 localized in the lower portion 28 inside the film of the imprint material 50 from the lower portion 28 toward the upper side (the template 40 side) is suppressed when the template 40 is caused to contact the imprint material 50.

As illustrated in FIG. 6D, for example, the light 61 is irradiated onto the imprint material 50 to form the imprint film 33 which is made of the imprint material 50 and includes the bottom portion 31 and the apical portion 32 that reflect the uneven configuration of the template 40.

At this time, by the reaction initiator 26 having a photo-crosslinkable structure in addition to the surfactant structure, crosslinking of the reaction initiator 26 is performed and the reaction initiator 26 is affixed to the lower portion 28 when irradiating the light 61 onto the imprint material 50. Thereby, the movement of the reaction initiator 26 toward the upper portion 34 due to the stress, etc., of the release of the template 40, etc., is suppressed.

Subsequently, as illustrated in FIG. 6E, the template 40 is released by being released from the cured imprint film 33.

After releasing the template 40 as illustrated in FIG. 5, the etching rate of the lower portion 28 is increased to be higher than the etching rate of a portion of the imprint film 33 excluding the lower portion 28 by causing the at least one selected from the acid and the base to be produced from the reaction initiator 26 by at least one selected from irradiating light onto the imprint film 33 and heating the imprint film 33 (step S230). In other words, a reaction is implemented to increase the etching rate of the bottom portion 31 to be higher than the etching rate of the apical portion 32.

Specifically, as illustrated in FIG. 6F, the foundation film 20 is heated to cause an acid 26a to be produced from the reaction initiator 26 inside the foundation film 20, diffuse the acid 26a into the bottom portion 31 of the imprint film 33, and introduce the acid 26a into the bottom portion 31 of the imprint film 33. The acid 26a is diffused into the bottom portion 31 by this heating.

In the bottom portion 31, the imprint film 33 is decomposed by the acid 26a. On the other hand, the acid 26a is not introduced into the apical portion 32. In other words, in the bottom portion 31, a reaction (e.g., a hydrolysis reaction) is performed to increase the etching rate of the bottom portion 31 to be higher than the etching rate of the apical portion 32.

As illustrated in FIG. 5 and FIG. 6G, the recessed portion of the imprint film 33 is removed (step S240). The bottom portion 31 of the imprint film 33 is removed by performing anisotropic dry etching such as, for example, oxygen plasma. At this time, the decrease of the height of the apical portion 32 is small because the etching rate of the apical portion 32 of the imprint film 33 is lower than that of the bottom portion 31.

As illustrated in FIG. 5 and FIG. 6H, the patterning film 10 is patterned using the protruding portion of the imprint film 33 (i.e., the remaining portion of the imprint film 33) as a mask (step S250). Because the apical portion height h2 is high in this embodiment, the imprint film 33 can exist until the end of the patterning of the patterning film 10; and the desired patterning of the patterning film 10 is possible.

After the patterning of the patterning film 10, oxygen plasma processing is performed; and the imprint film 33 is removed by ashing. Thereby, the pattern of the silicon oxide film is formed.

In the description recited above, a material in which an acryloyl group is bonded to a portion of the structure can be used as the reaction initiator 26. In such a case, the upper portion 34 inside the film of the imprint material 50 (including, for example, an acrylic monomer and a radical initiator) chemically bonds to the reaction initiator 26 when performing the light irradiation to cure the imprint material 50 in the state in which the template 40 is caused to contact the imprint material 50. Thereby, the adhesion between the lower portion 28 and the upper portion 34 of the imprint material 50 is increased. A high adhesion between the imprint material 50 and the patterning film 10 can be ensured by, for example, hydrogen bonding between the reaction initiator 26 and the patterning film 10. It is more desirable for an acryloyl group to be introduced into the reaction initiator 26.

Thus, the reaction initiator 26 may function as an adhesion improver in addition to functioning as the reaction initiator to produce the at least one selected from the acid and the base. Thereby, a dedicated film for increasing the adhesion can be eliminated while efficiently removing the residual film.

Although the etching resistance of the reaction initiator 26 is increased in the case where the reaction initiator 26 is, for example, photo-crosslinkable, the etching rate of the lower portion 28 (the bottom portion 31) is higher than the etching rate of the apical portion 32 because the one other material 22 (the resin, etc.) included in the lower portion 28, in which the reaction initiator 26 is localized, is decomposed by the at least one selected from the acid and the base produced from the reaction initiator 26.

Thus, the imprint material 50 according to the embodiment may be an imprint material which is, for example, coated onto a patterning film, usable in a pattern formation method by imprinting, and in which the at least one selected from the acid and the base is produced by at least one selected from irradiating light and heating, where the imprint material includes a reaction initiator having a surfactant structure. Further, this imprint material may be photo-crosslinkable. This imprint material also may include an acryloyl group. Herein, the acryloyl group is taken to include the methacryloyl group.

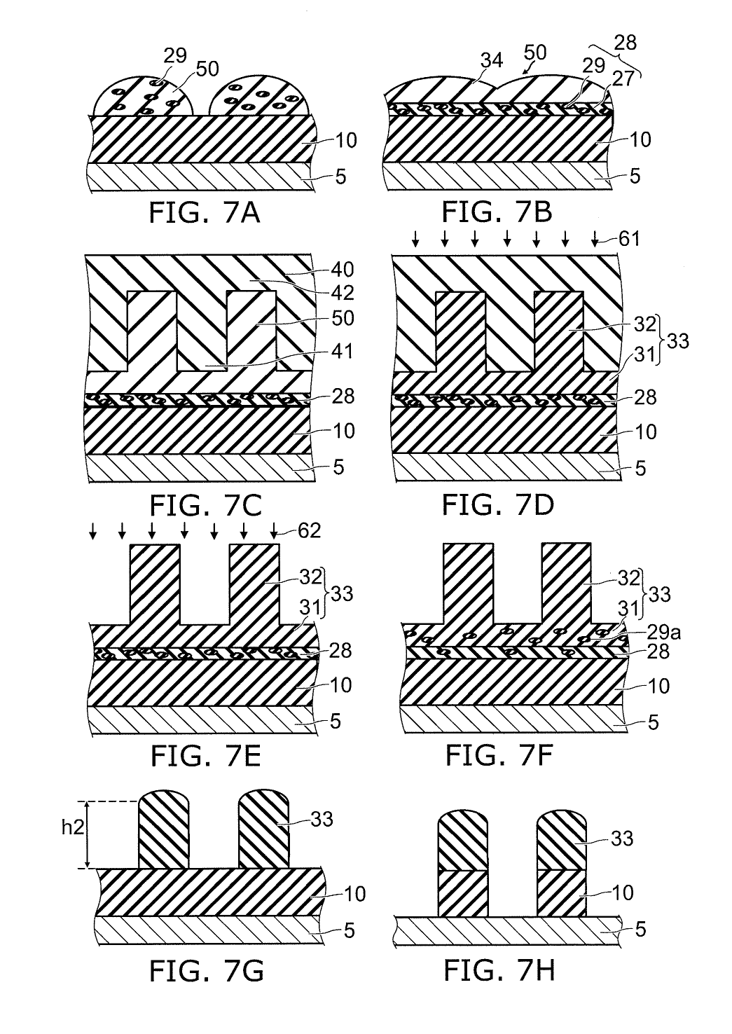

FIG. 7A to FIG. 7H are schematic cross-sectional views illustrating another pattern formation method according to the second embodiment.

In this pattern formation method according to this embodiment, a substance that produces the at least one selected from the acid and the base by being irradiated with light is used as the reaction initiator.

As illustrated in FIG. 7A, the imprint material 50 including a reaction initiator 29 configured to produce the at least one selected from the acid and the base is coated onto the patterning film 10 (step S210). Bis(t-butylsulfonyl)diazomethane, for example, is used as the reaction initiator 29.

Then, as illustrated in FIG. 7B, the imprint material 50 coated onto the patterning film 10 is separated into the lower portion 28 and the upper portion 34. In other words, layer separation is performed.

As illustrated in FIG. 7C, the template 40 is caused to contact the imprint material 50 in the state in which the reaction initiator 29 is localized in the lower portion 28 of the imprint material 50 on the patterning film 10 side.

As illustrated in FIG. 7D, for example, the imprint film 33 is formed by irradiating the light 61 onto the imprint material 50, where the imprint film 33 is made of the imprint material 50 and includes the bottom portion 31 and the apical portion 32 that reflect the uneven configuration of the template 40. In other words, step S220 is implemented.

As illustrated in FIG. 7E, the template 40 is released by being released from the cured imprint film 33.

Configurations and methods similar to those described in regard to FIG. 6A to FIG. 6E can be applied for the processes from the layer separation of the imprint material 50 to the release of the template 40 recited above, and a description is therefore omitted.

As illustrated in FIG. 7F, an acid 29a is produced from the reaction initiator 29 inside the foundation film 20 by irradiating the light 62 onto the foundation film 20. The light 62 at this time may include light having a wavelength, for example, not more than 270 nm. Specifically, the wavelength of the light 62 may be 248 nm.

Subsequently, the acid 29a produced from the reaction initiator 29 is diffused into the bottom portion 31 by heating the patterning body for 1 minute at a temperature of 110° C. in air.

In the bottom portion 31, the imprint film 33 is decomposed by the acid 29a. On the other hand, the acid 29a is not introduced into the apical portion 32. In other words, in the bottom portion 31, a reaction (e.g., a hydrolysis reaction) is performed to increase the etching rate of the bottom portion 31 to be higher than the etching rate of the apical portion 32. In other words, step S230 is implemented.

As illustrated in FIG. 7G, anisotropic dry etching such as, for example, oxygen plasma is performed; and the bottom portion 31 of the imprint film 33 is removed. In other words, step S240 is implemented.

The decrease of the height of the apical portion 32 is small because the etching rate of the apical portion 32 of the imprint film 33 is lower than that of the bottom portion 31.

As illustrated in FIG. 7H, the patterning film 10 is patterned using the imprint film 33 as a mask. In other words, step S250 is implemented.

Although the thickness of the imprint film 33 used as the mask also is reduced during the patterning of the patterning film 10 of the desired thickness, in this embodiment, the imprint film 33 can exist until the end of the patterning of the patterning film 10 and the desired patterning of the patterning film 10 is possible because the apical portion height h2 is high.

After the patterning of the patterning film 10, oxygen plasma processing is performed; and the imprint film 33 is removed by ashing. Thereby, the pattern of the silicon oxide film is formed.

Although specific examples of materials that produce an acid are described as the reaction initiators 21, 25, 26, and 29 in the specific examples described above, a material that produces a base may be used as the reaction initiators 21, 25, 26, and 29. Materials that produce a base include, for example, N-(2-nitrobenzyloxycarbonyl)imidazole, N-(3-nitrobenzyloxycarbonyl)imidazole, N-(4-nitrobenzyloxycarbonyl)imidazole, N-(5-methyl-2-nitrobenzyloxycarbonyl)imidazole, N-(4-chloro-2-nitrobenzylcarbonyl)imidazole, etc. However, the embodiment is not limited thereto. Any material that produces a base can be used as the reaction initiator.

According to the embodiments of the invention, a pattern formation method and an imprint material capable of efficiently removing the residual film can be provided.

In the specification of the application, “perpendicular” and “parallel” refer to not only strictly perpendicular and strictly parallel but also include, for example, the fluctuation due to manufacturing processes, etc. It is sufficient to be substantially perpendicular and substantially parallel.

Hereinabove, exemplary embodiments of the invention are described with reference to specific examples. However, the invention is not limited to these specific examples. For example, one skilled in the art may similarly practice the invention by appropriately selecting specific configurations of components such as substrates, patterning films, imprint materials, foundation films, reaction initiators, templates, etc., used in pattern formation methods from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all pattern formation methods practicable by an appropriate design modification by one skilled in the art based on the pattern formation methods described above as embodiments of the invention also are within the scope of the invention to the extent that the purport of the invention is included.

Furthermore, various modifications and alterations within the spirit of the invention will be readily apparent to those skilled in the art. All such modifications and alterations should therefore be seen as within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modification as would fall within the scope and spirit of the inventions.

What is claimed is:

1. A pattern formation method, comprising;
forming a foundation film on a patterning film, the foundation film including a reaction initiator configured to produce at least one selected from an acid and a base;
forming an imprint film having an uneven configuration by coating an imprint material onto the foundation film and by causing a template to contact the imprint material;
increasing an etching rate of a first portion of the imprint film to be higher than an etching rate of a second portion of the imprint film by causing the at least one selected from the acid and the base to be produced from the reaction initiator and by introducing the at least one selected from the acid and the base into the first portion, the first portion being a portion of the imprint film on the foundation film side, the second portion being a portion of the imprint film excluding the first portion;
removing a recessed portion of the uneven configuration of the imprint film; and
patterning the patterning film using a protruding portion of the uneven configuration of the imprint film as a mask.

2. The method according to claim 1, wherein the increasing the etching rate of the first portion includes heating the foundation film to cause the at least one selected from the acid and the base to be produced and to move the at least one selected from the acid and the base into the imprint film.

3. The method according to claim 1, wherein the forming of the imprint film includes curing the imprint material by irradiating light onto the imprint material in the state of the template contacting the imprint material.

4. The method according to claim 1, wherein the increasing of the etching rate of the first portion includes:
irradiating light onto the foundation film to cause the at least one selected from the acid and the base to be produced; and
heating the foundation film to move the at least one selected from the acid and the base into the imprint film after the irradiating the light onto the foundation film.

5. The method according to claim 4, wherein
the forming of the imprint film includes irradiating light onto the imprint material, and
a wavelength of the light irradiated in the irradiating the light onto the foundation film is shorter than a wavelength of the light irradiated onto the imprint material in the forming of the imprint film.

6. The method according to claim 1, wherein the reaction initiator includes at least one selected from a material configured to produce the acid by heating, a material configured to produce the acid by irradiating light, a material configured to produce the base by heating, and a material configured to produce the base by irradiating light.

7. The method according to claim 1, wherein the reaction initiator includes an aromatic sulfonium salt.

8. The method according to claim 1, wherein the foundation film further includes at least one selected from a solvent and an adhesion improver.

9. The method according to claim 1, wherein a thickness of the foundation film is not less than 1 nanometer and not more than 5 nanometers.

10. The method according to claim 1, wherein the imprint material includes a photocurable resin.

11. The method according to claim 1, wherein the increasing of the etching rate of the first includes the at least one selected from the acid and the base decomposing a chemical bond included in the first portion.

12. The method according to claim 1, wherein the increasing of the etching rate of the first portion includes the acid decomposing an ester bond included in the first portion.

13. The method according to claim 1, wherein the removing of the recessed portion includes performing anisotropic dry etching.

14. The method according to claim 1, wherein the reaction initiator includes bis(t-butylsulfonyl)diazomethane.

15. A pattern formation method, comprising:
coating an imprint material onto a patterning film, the imprint material including a reaction initiator configured to produce at least one selected from an acid and a base;
forming an imprint film having an uneven configuration by causing a template to contact the imprint material in a state of the reaction initiator being localized in a lower portion of the imprint material on the patterning film side;
increasing an etching rate of the lower portion to be higher than an etching rate of a portion of the imprint film excluding the lower portion by causing the at least one selected from the acid and the base to be produced from the reaction initiator by at least one selected from irradiating light onto the imprint film and heating the imprint film;
removing a recessed portion of the uneven configuration of the imprint film; and
patterning the patterning film using a protruding portion of the uneven configuration of the imprint film as a mask.

16. The method according to claim 15, wherein the forming of the imprint film includes curing the imprint material by irradiating light onto the imprint material in the state of the template contacting the imprint material.

17. The method according to claim 15, wherein the reaction initiator includes a material having a surfactant structure including a hydrophilic portion chemically bonded to a hydrophobic portion.

18. The method according to claim 15, wherein a surface of the template caused to contact the imprint material has a layer processed for low surface tension.

19. The method according to claim 15, wherein the reaction initiator includes a material including an acryloyl group.

20. An imprint material coated onto a patterning film and usable in a pattern formation method by imprinting, the imprint material comprising;
a reaction initiator, the reaction initiator having a surfactant structure and being configured to produce at least one selected from an acid and a base by at least one selected from irradiating light and heating.
